(19) United States
(12) Patent Application Publication
(10) Pub. No.: US 2018/0256915 A1
(43) Pub. Date: Sep. 13, 2018

(54) METHOD OF USING A LIGHT-BASED DRUG DISPENSING SYSTEM TO SIMULATE THE OPERATION OF A HUMAN PANCREAS

(71) Applicants: Rama Madugula, Royal Oak, MI (US); Anup Sabharwal, Miami, FL (US); Sankar Majeti, Rochester Hills, MI (US)

(72) Inventors: Rama Madugula, Royal Oak, MI (US); Anup Sabharwal, Miami, FL (US); Sankar Majeti, Rochester Hills, MI (US); Murty Madugula, Rochester Hills, MI (US)

(73) Assignee: Midwest Business Associates, Inc., Rochester Hills, MI (US)

(21) Appl. No.: 15/975,973

(22) Filed: May 10, 2018

Related U.S. Application Data
(62) Division of application No. 15/247,796, filed on Aug. 25, 2016.

(51) Int. Cl. A61N 5/06; A61K 31/64; A61K 45/06; A61M 5/14; G16H 20/10; G08B 21/04

(52) U.S. Cl. CPC A61N 5/062; A61K 31/64; A61K 45/06; A61M 5/14; G16H 20/10; G08B 21/0453; A61M 2205/52; A61M 2205/3584; A61M 2205/583; A61N 2005/0626; A61N 2005/0652; A61N 2005/0663; A61M 2205/18

(57) ABSTRACT

Disclosed is a light-based method for dispensing drug formulations that simulates the operation of a human pancreas. Drug formulations dispensable by the method contain at least one synthetic molecular switch compound whose structure and dosage amounts are controllable by light energy and based on both light wavelength and light intensity. The method dispenses drug formulations based on light generated by a set of LED arrays that includes blue LED's; the light is detected by photoreceivers within a drug depot matrix port that contains the drug formulations. The method is designed such that multiple drug formulations used simultaneously are each controlled by a separate LED array and by separate photoreceivers operating at separate wavelengths such that the drug formulations, LED arrays, photoreceivers and wavelengths do not interfere with each other.

### CROSS REFERENCE TO RELATED APPLICATIONS

The present invention is a divisional from U.S. patent application Ser. No. 15/247,796, filed 25 Aug. 2016, entitled “Light-Based Drug Dispensing System to Simulate the Operation of a Human Pancreas”, by Rama Madugula, et al., which is, in turn, a continuation-in-part (CIP) of U.S. patent application Ser. No. 14/724,854, filed 29 May 2015, entitled “System for Medical Device Software Alert Hierarchy Management”, by Rama Madugula, et al.

### BACKGROUND OF THE INVENTION

### a. Field of the Invention

The present invention generally pertains to methods used to monitor the health of a patient and more particularly to methods that use a computer system to deliver drug formulation to a patient suffering from diabetes.

### b. Description of the Background

Patients with chronic health conditions, such as diabetes, are often medicated using a system that includes at least one sensor to measure important levels (such as blood glucose), a computer program that decides when a patient should receive drug formulation based on those levels and how much drug formulation should be given, and at least one method of dispensing the drug formulation into a patient's bloodstream. Sensors, programs and dispensing methods in such a system are constantly under development to improve their usefulness and reduce cost. However, such systems are usually mechanically based, especially the method for dispensing drug formulation.

It would therefore be advantageous to have a non-mechanical method for dispensing drug formulation that would simulate the operation of a human pancreas. It would be further advantageous if such a method contained a computer program that could monitor the blood glucose level of a diabetic patient and provide drug formulation to the patient through use of an LED array that is tuned to operate at a desired wavelength and would provide the drug formulation when light of a certain wavelength and intensity is generated.

### SUMMARY OF THE INVENTION

The present invention overcomes the disadvantages and limitations of the prior art by creating a light-based method for dispensing drug formulations that simulates the operation of a human pancreas. Drug formulations dispensable by the method contain at least one synthetic molecular switch compound whose structure and dosage amounts are controllable by light energy and based on both light wavelength and light intensity. The method dispenses drug formulations based on light generated by a set of LED arrays that includes blue LED's; the light is detected by photoreceivers within a drug depot matrix port that contains the drug formulations. The method is designed such that multiple drug formulations used simultaneously are each controlled by a separate LED array and by separate photoreceivers operating at separate wavelengths such that the drug formulations, LED arrays, photoreceivers and wavelengths do not interfere with each other.

The present invention may thus comprise a method of dispensing drug formulations by using a light-based system to simulate the operation of a human pancreas, comprising: ensuring that a drug formulation contains at least one synthetic molecular switch compound whose structure and dosage amounts are controllable by light energy and based on both light wavelength and light intensity; loading at least one of the drug formulations into a drug depot matrix port that includes at least one set of photoreceivers that detects light from at least one set of LED arrays; setting at least one set of said LED arrays to generate at least one light beam of a wavelength and intensity that controls the dosage of the drug formulation given to a patient; monitoring of the physical condition of the light-based system; monitoring of the physical condition of the patient; and, using a computer program to monitor the amount of a drug formulation in a drug depot matrix port, control the amount of a drug formulation given to a patient, monitor the amount of light detected by the photoreceivers, monitor the operating temperature and control the operation of the LED arrays, monitor information received from sensors used by the patient, generate and send alerts to problems detected with the patient, and generate and send alerts to problems detected within the light-based system.

The drug formulation may be given to a patient either orally or through the drug depot matrix port. The drug formulation may be a sulfonylurea compound (such as JB253), a diabetes drug formulation, insulin, or an anti-inflammatory compound. The drug depot matrix port may be located outside the body of a patient, may be a subcutaneous drug reservoir within the body of a patient, or may be an intramuscular drug reservoir within the body of a patient. The computer program may control the set of LED arrays by setting the pulse width modulation duty cycle or by setting the maximum amount of LED activation within a given time period. The set of LED arrays may be located inside or outside the body of a patient, and the set of LED arrays may contain blue LED's. Multiple drug formulations used simultaneously within the drug depot matrix port are each controlled by a separate LED array and by separate photoreceivers operating at separate wavelengths such that the drug formulations, LED arrays, photoreceivers and wavelengths do not interfere with each other. The alerts generated and sent for problems detected with a patient, as well as the alerts generated and sent for problems detected within the light-based system, include visual displays, auditory alarms, outputs to a message module, or wireless communications methods.

### BRIEF DESCRIPTION OF THE DRAWINGS

In the drawings,

FIG. 1 is a drawing of the system of the present invention.

FIG. 2 is an operational flowchart of the present invention.

### DETAILED DESCRIPTION OF THE INVENTION

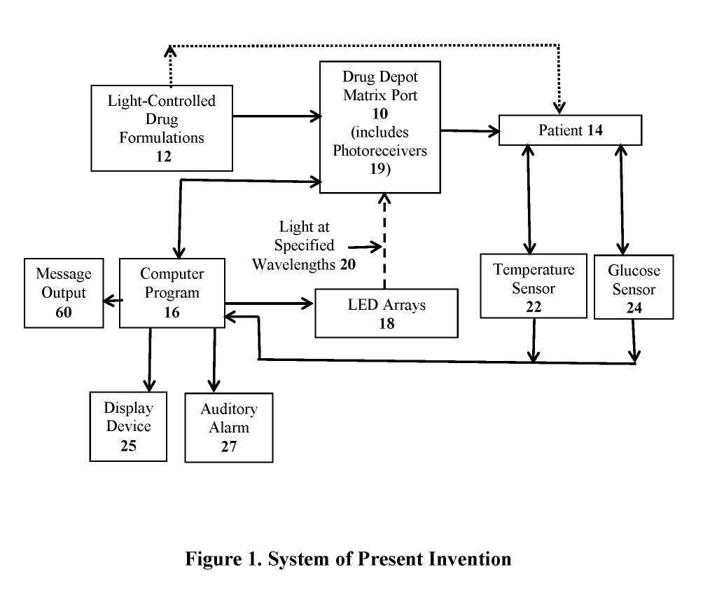

FIG. 1 is a drawing of the system of the present invention, which includes a drug depot matrix port 10 that receives and holds at least one light-controlled drug formulation 12 that may be given to a patient 14. The drug depot matrix port 10 may be a subcutaneous or intramuscular drug reservoir within the body of a patient and may hold at least one light-controlled drug formulation 12 that is released into the patient's body in controlled doses via controlled amounts of light energy from at least one set of LED arrays 18.

In some embodiments, the light-controlled drug formulations 12 may be given orally to the patient 14, as indicated by the dashed line in FIG. 1. In optimum embodiments, the light-controlled drug formulations 12 may each contain a synthetic molecular switch compound whose structure can be controlled by a computer program 16 via a light-emitting diode (LED) of a specified wavelength within the LED arrays 18.

The computer program 16 may be set by a physician to provide feedback and control the sets of LED arrays 18 to generate light at specified wavelengths 20 that may be received by a set of photoreceivers 19 within the drug depot matrix port 10. The photoreceivers 19 may be located within the drug depot matrix port 10 to ensure that the light 20 reaches the light-controlled drug formulations 12 within the drug depot matrix port 10. Sensors, including a temperature sensor 22 and glucose sensor 24, may be connected to the patient 14 either inside or outside the patient's body, and all sensor outputs may be provided to the computer program 16, along with the outputs of the photoreceivers 19. Both the drug depot matrix port 10 and sets of LED arrays 18 may be located either inside or outside the patient's body.

The computer program 16 may be set by a physician to control the set of LED arrays 18, including the pulse width modulation duty cycle and the maximum amount of LED activation within a given time period. The computer program 16 may also be set by a physician to monitor related parameters as needed, including the temperature range of the LED arrays 18 as registered by the temperature sensor 22 and the range of glucose levels registered by the glucose sensor 24 at which all LED's in the LED arrays 18 should be deactivated. As a safety mechanism, the computer program 16 may also be set by a physician to limit the total amount of time the LED arrays 18 may be activated within a defined period to protect a patient from an overdose of a light-controlled drug formulation 12.

Information from the computer program 16 may be viewed on a display device 25 and output to a message module 60 to allow notification of the patient, family members and caregivers of any parameter outside established ranges. Any parameter outside established ranges may cause the computer program 16 to generate an auditory alarm 27 that something in the system of FIG. 1 needs to be repaired. An auditory alarm 27 may also be generated when the photoreceivers 19 detect that light from the LED arrays 18 is not reaching the light-controlled drug formulations 12 within the drug depot matrix port 10.

In optimum embodiments of the present invention, the LED's in the LED arrays 18 may be blue in color, and each light-controlled drug formulation 12 may be controlled by a separate LED array 18 and photoreceivers 19 that operate at different wavelengths 20 such that the light-controlled drug formulations 12, LED arrays 18, photoreceivers 19 and wavelengths 20 do not interfere with each other. The light-controlled drug formulations 12 may include (but are not limited to) such drug formulations as a sulfonylurea compound (such as JB253), a diabetes drug formulation, insulin, an anti-inflammatory compound or any other drug formulation deemed medically beneficial for a patient.

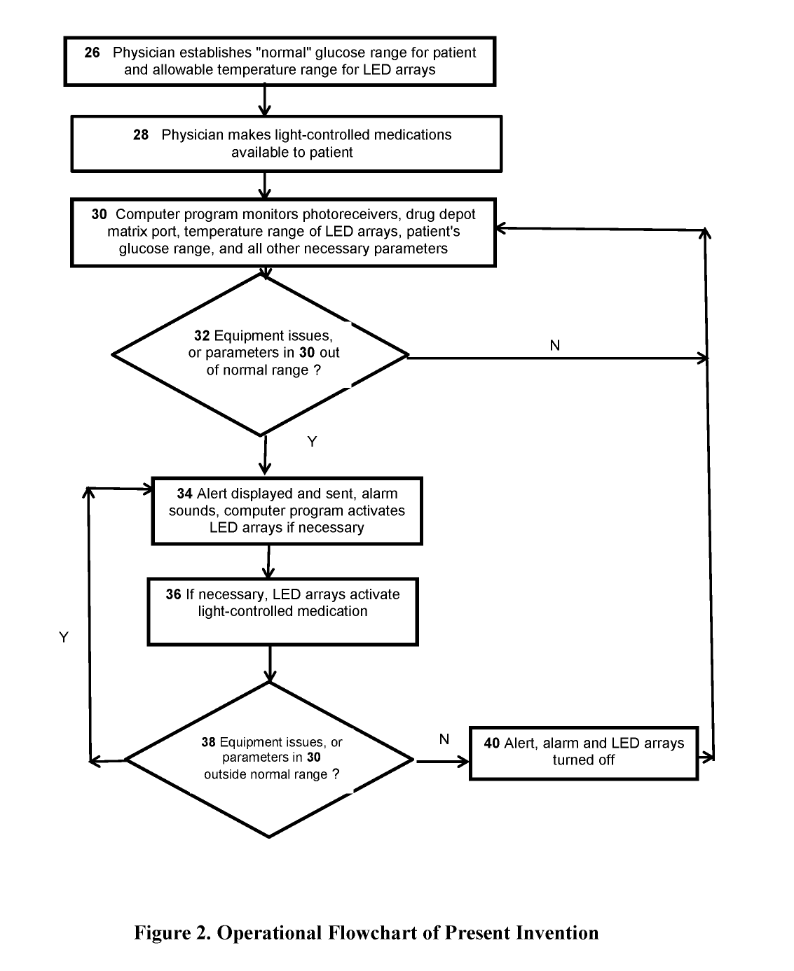

FIG. 2 illustrates an operational flowchart for the present invention. A physician establishes a range of allowable temperatures for the LED arrays 18 such that the LED arrays 18 do not burn the patient 14. The physician also establishes a range of “normal” glucose levels 26 individualized for each patient, along with a range for any other sensors that may be used in the system (such as a heart monitor, body temperature sensor or blood pressure sensor). The physician 28 then makes the light-controlled drug formulations 12 (referring to FIG. 1) available to the patient by either placing the light-controlled drug formulations 12 in the drug depot matrix port 10 or giving the light-controlled drug formulations 12 orally to the patient 14. As long as the photoreceivers 19 are working properly, the drug depot matrix port 10 contains sufficient light-controlled drug formulations 12, and the patient's temperature range, glucose level and other parameters are within normal ranges as detected by the temperature sensor 22, glucose sensor 24 and other sensors in the system, the present invention may remain in a quiescent state 30, monitored by the computer program 16.

Whenever the photoreceivers 19 detect a lack of light to the light-controlled drug formulations 12, the drug depot matrix port 10 needs more drug formulation, the LED array 18 is hot enough to burn the patient's skin, or a sensor level 32 is outside of the range calibrated by the physician, the computer program 34 may detect the situation and, as necessary, send an alert to the display device 25 (referring to FIG. 1) and message module 60, send an alert to the auditory alarm 27 to create a sound, and/or send an activation command to one or more specific LED arrays 18. These activated LED arrays 18 may then activate a combination of LED's 36 to generate light at specified wavelengths and intensities corresponding to at least one light-controlled drug formulation 12. The specific wavelengths and intensities of the light 20 (referring to FIG. 1) activate the light-controlled drug formulations 12, which are then delivered to the patient in an appropriate dosage calculated by the physician and computer program 16 from data provided by the glucose sensor 24 and other sensors in the system. The light-controlled drug formulations 12 may be administered until the glucose sensor and other sensors indicate 38 that the patient's glucose and other levels are again within the “normal” range. The photoreceivers 19 may again detect light being sent to the light-controlled drug formulations 12 and the drug depot matrix port 10 may be replenished as needed, at which point the computer program 16 turns off all alerts to the display device 25, message module 60 and auditory alarm 27. The computer program 16 may also send a message to the activated LED arrays 40 that turns off the lights and ends the dosage of the light-controlled drug formulations 12.

Note that even in cases where the patient 14 has received at least one light-controlled drug formulation 12 orally, the light 20 may still control the light-controlled drug formulation within the body of the patient 14 by directing operation of the LED arrays 18 as described above.

In all embodiments of the present invention, alerts may be sent by common electronic communications methods, including wireless methods, that may include wi-fi, e-mail, texting, cell phone and Bluetooth. Such alerts may be seen on a remote device such as a smartphone, wrist watch or other display in a remote location and may notify all interested parties, such as a patient, caregiver, physician and hospital, of a patient's health issues.

The present invention reduces the cost of a patient's treatment regimen and improves a patient's long-term health.

The foregoing description of the invention has been presented for purposes of illustration and description. It is not intended to be exhaustive or to limit the invention to the precise form disclosed, and other modifications and variations may be possible in light of the above teachings. The embodiments were chosen and described in order to best explain the principles of the invention and its practical application to thereby enable others skilled in the art to best utilize the invention in various embodiments and various modifications as are suited to the particular use contemplated. It is intended that the appended claims be construed to include other alternative embodiments of the invention except insofar as limited by the prior art.

What is claimed is:

1. A method of dispensing drug formulations by using a light-based system to simulate the operation of a human pancreas, comprising:
ensuring that said drug formulation contains at least one synthetic molecular switch compound whose structure and dosage amounts are controllable by light energy and based on both light wavelength and light intensity;
loading at least one of said drug formulation into a drug depot matrix port that includes at least one set of photoreceivers that detects light from at least one set of LED arrays;
setting at least one set of said LED arrays to generate at least one light beam of a wavelength and intensity that controls the dosage of said drug formulation given to a patient;
monitoring of the physical condition of said light-based system;
monitoring of the physical condition of said patient; and,
using a computer program to monitor the amount of said drug formulation in said drug depot matrix port, control the amount of said drug formulation given to said patient, monitor the amount of light detected by said photoreceivers, monitor the operating temperature and control the operation of said LED arrays, monitor information received from sensors used by said patient, generate and send alerts to problems detected with said patient, and generate and send alerts to problems detected within said light-based system.

2. The method of claim 1, wherein said drug formulation is given to said patient either orally or through said drug depot matrix port.

3. The method of claim 1, wherein said drug formulation is a sulfonylurea compound, a diabetes drug formulation, insulin, or an anti-inflammatory compound.

4. The light-based method of claim 3, wherein said sulfonylurea compound is JB253.

5. The method of claim 1, wherein said drug depot matrix port is located outside the body of said patient, is a subcutaneous drug reservoir within the body of said patient, or is an intramuscular drug reservoir within the body of said patient.

6. The method of claim 1, wherein said computer program may control said set of LED arrays by setting the pulse width modulation duty cycle or by setting the maximum amount of LED activation within a given time period.

7. The method of claim 1, wherein said set of LED arrays is located inside the body of said patient or outside the body of said patient.

8. The method of claim 1, wherein said set of LED arrays contain LED's that are blue in color.

9. The method of claim 1, wherein multiple drug formulations used simultaneously within said drug depot matrix port are each controlled by a separate LED array and by separate photoreceivers operating at separate wavelengths such that said drug formulations, said LED arrays, said photoreceivers and said wavelengths do not interfere with each other.

10. The method of claim 1, wherein the alerts generated and sent for problems detected with said patient and the alerts generated and sent for problems detected within said light-based system include visual displays, auditory alarms, outputs to a message module, or wireless communications methods.
